(19) United States
(12) Patent Application Publication
(10) Pub. No.: US 2023/0281362 A1
(43) Pub. Date: Sep. 7, 2023

(54) PARAMETER AND STATE INITIALIZATION FOR MODEL TRAINING

(71) Applicant: X Development LLC, Mountain View, CA (US)

(72) Inventors: Nicholas Ruggero, Las Vegas, NV (US); Federico Vaggi, Seattle, WA (US); Mohammad Mahdi Shafiei, Cupertino, CA (US); Joseph Dale, Sunnyvale, CA (US)

(73) Assignee: X Development LLC, Mountain View, CA (US)

(21) Appl. No.: 17/649,472

(22) Filed: Jan. 31, 2022

(51) Int. Cl. G06F 30/27

(52) U.S. Cl. CPC G06F 30/27

(57) ABSTRACT

A set of conditions is defined that to be simulated via execution of a machine-learning model. For each condition, a set of learnable condition-specific parameters is identified to configure a model architecture. A first learnable condition-specific parameter associated with a first condition of the set of conditions can be identified a shared or global parameter that is to have a same value as at least another learnable condition-specific parameter (associated with another condition). One or more parameter data structures can be configured with parameter values for the sets of condition-specific parameters for the sets of conditions, where the configuration imposes a constraint that a value for the first condition-specific parameter and the at least one value for the at least one other condition-specific parameter are the same. The machine-learning model can be trained using the configured parameter data structure(s).

### BACKGROUND

Training machine-learning models can be very time intensive and can use a substantial amount of computing resources (e.g., CPU cycles, memory, etc.). This resource commitment typically scales with the complexity of models. Similarly, the size of a training data set required for training typically scales with model complexity.

However, complex models can be very valuable. For example, it can be useful to have a single model that can simulate how a given system performs in different conditions. This can facilitate identifying (for example) which parts of the system are important to compensating deficiencies present in particular conditions, to understanding how a system dynamically adjusts to a condition change, et.

Thus, it would be advantageous to identify a technique to support training complex models with reduced resource and training-data commitment.

### SUMMARY

In some embodiments, a set of conditions is defined that to be simulated via execution of a machine-learning model. For each condition, a set of learnable condition-specific parameters is identified to configure a model architecture. A first learnable condition-specific parameter associated with a first condition of the set of conditions can be identified a shared or global parameter that is to have a same value as at least another learnable condition-specific parameter (associated with another condition). One or more parameter data structures can be configured with parameter values for the sets of condition-specific parameters for the sets of conditions, where the configuration imposes a constraint that a value for the first condition-specific parameter and the at least one value for the at least one other condition-specific parameter are the same. The machine-learning model can be trained using the configured parameter data structure(s). The trained machine-learning model can be executed by processing another data set.

Configuring the one or more parameter data structures can include generating an initial version of a parameter data structure of the one or more parameter data structures to include a value for each of the sets of learnable condition-specific parameters of the set of conditions; identifying an initial value to initially define the shared or global parameter; and generating a modified version of the parameter data structure to replace an initial version of the at least one other learnable condition-specific parameter with the initial value.

Training the machine-learning model can be performed using a loss function that relates loss to values of a set of unique learnable parameters, and wherein the quantity of unique learnable parameters in the set of unique learnable parameters is less than a quantity of parameters represented in the one or more parameter data structures.

Training the machine-learning model can include: calculating a loss function, wherein the loss function associates a particular loss with values of a set of learnable parameters, wherein the set of learnable parameters includes a particular learnable parameter corresponding to the first learnable condition-specific parameter and the at least one other learnable condition-specific parameter; identifying a new set of values for the set of learnable parameters using the loss function, wherein the new set of values includes a new value for the particular learnable parameter; and updating the one or more parameter data structures using the new set of values for the set of learnable parameters, wherein the updating includes setting each of the at least one value for the at least one other condition-specific parameter and the value for the first condition-specific parameter to the new value.

The first learnable condition-specific parameter can be a shared parameter, the combination of the first condition and each corresponding other condition associated with the at least one other learnable condition-specific parameter are an incomplete subset of the set of conditions, and the method can further include stipulating that a different learnable condition-specific parameter is a global parameter that is to have a same value across all conditions in the set of conditions; wherein configuring the one or more parameter data structures imposes a constraint that values for parameters corresponding to the global parameter are to be the same across conditions.

The machine-learning model can be a model to simulate a biological cell, and at least one of the set of conditions can correspond to a simulation where a particular gene is missing or inactive.

The machine-learning model can be a model to simulate a biological cell, and at least one of the set of conditions can correspond to a simulation where a particular reagent is added to a medium external to the biological cell.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an interaction system for configuring and using a simulation to facilitate subsequent experiment configurations according to some embodiments of the invention;

FIG. 2 shows a representation of modules representing distinct biological functions according to an embodiment of the invention;

FIG. 3 shows a simulation controller that dynamically integrates results generated by different types of models to simulate higher-level states and reactions according to some embodiments of the invention;

FIG. 4 shows a process for dynamically synthesizing results generated by multiple simulators to simulate higher-level results according to an embodiment of the invention;

FIG. 5 shows a module-specific simulation controller to simulate states and reactions according to some embodiments of the invention;

FIG. 6 shows a process for using a simulator to generate metabolite time-course data according to an embodiment of the invention;

FIG. 7 shows a box diagram of an exemplary computing system for building a machine learning model; and

FIG. 8 shows a flowchart of an exemplary process for training and using a machine learning model for use across different conditions.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

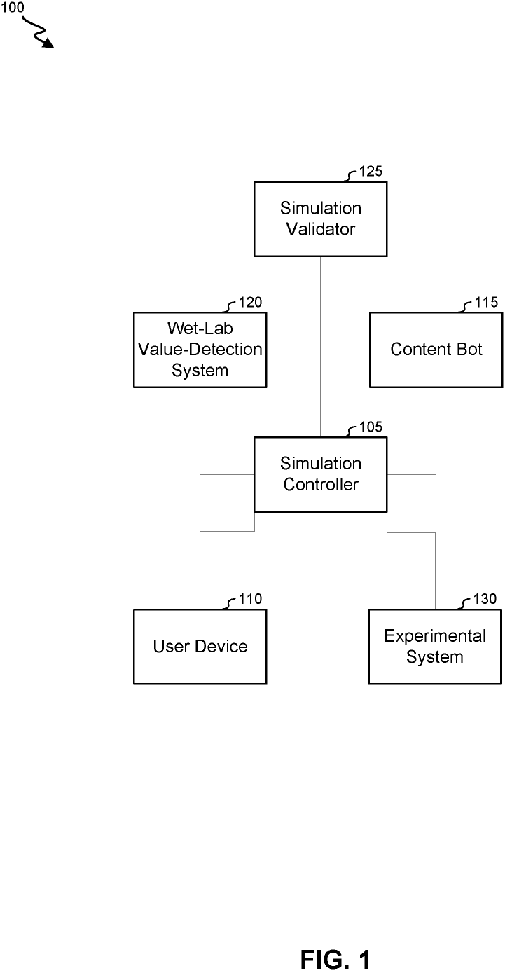

FIG. 1 shows an interaction system 100 for configuring instances or versions of a model and using a simulation to facilitate subsequent experiment configurations (e.g., simulation of a biological system’s response to a new demand) according to various embodiments. Each instance of the models may have a combination of modules, perturbations (such as knockouts), and may be built using a particular set of experimental data. In order to facilitate the configuring of a model (e.g., a biological system) and simulate an outcome of the model, the interaction system 100 can include one or more components, each of which can include (for example) one or more servers, one or more computers and/or one or more mobile devices. In some instances, two or more of the components can be included in a same server, same server system, same computer, etc. Interaction system 100 can include one or more networks (e.g., a wired network, a wireless network, the Internet, a local area network, a wide area network, a short-range network, etc.), such that each component in the interaction system 100 can communicate with one or more other components in the interaction system 100.

Interaction system 100 can include a simulation controller 105 that defines, generates, updates and/or executes each of one or more simulations. A simulation can be configured to simulate dynamic progression through states, a time-evolved state of a model of a biological system and/or a steady state based on an iterative module-based assessment. It will be appreciated that identifying a steady-state and/or balanced solution for a module at a given time step need not indicate that a steady-state and/or balanced solution has been, can be or will be identified for the model in general (e.g., as metabolites produced and/or consumed at one module may further be produced and/or consumed at another module that need not be configured for balancing fluxes).

A given model can be used to generate and run any number of simulations. Differing initial conditions and/or differing automatically generated values in stochastic portions of the simulation (e.g., generated using a pseudo-random number generation technique, a stochastic pull from a distribution, etc.) can result in different output results of different simulations. The biological system model can be made up of one or more modules, and during a simulation run, each module is run independently and passes results back up to the biological system model level. More specifically, the biological system (e.g., a whole cell) may be modeled in accordance with a coordinated operation of multiple modules that represent structure(s) and/or function(s) of the biological system. Each module may be defined to execute independently, except that a shared set of state values (e.g., a state vector) maintained at the biological system model level may be used and accessed by multiple modules at each time point.

In some instances, each module of the biological system is configured to advance across iterations (e.g., time points) using one or more physiological and/or physics-based models (e.g., flux balance analysis (FBA), template synthesis, bulk-mass flow analysis, constant non-specific degradation, empirical analysis, etc.). The module-specific iteration processing can further be based on one or more module-specific state values (as determined based on an initial definition for an initial iteration processing or a result of a previous iteration processing for a subsequent iteration processing). The module-specific iteration processing can further be based on one or more parameters defined for the module that are fixed and/or static across iterations.

Simulation controller 105 can generate simulation configurations using one or more inputs received from a user device 110. For example, simulation controller 105 may generate an interface (or may at least partly define specifications for an interface) that is to be availed and/or transmitted to user device 110 and to include input fields configured to receive inputs that correspond to a selection of (for example) one or more modules to be used for a given biological system model, a model type to be used for each of the one or more modules, one or more parameters that are to be effected by a given module’s model and used during execution, and/or one or more initial state-value definitions that are to be used by a given module’s model and used during execution. In some instances, the interface identifies a default value for each of one, more or all parameters of the model and for each of one, more or all of the initial-state values of the model and is configured to receive a modification to a subset or all of the parameters and/or initial-state values for which a default value was identified. In some instances, modifying a default initial-state value and/or parameter can correspond to a perturbation of performance of a corresponding module and/or the biological system.

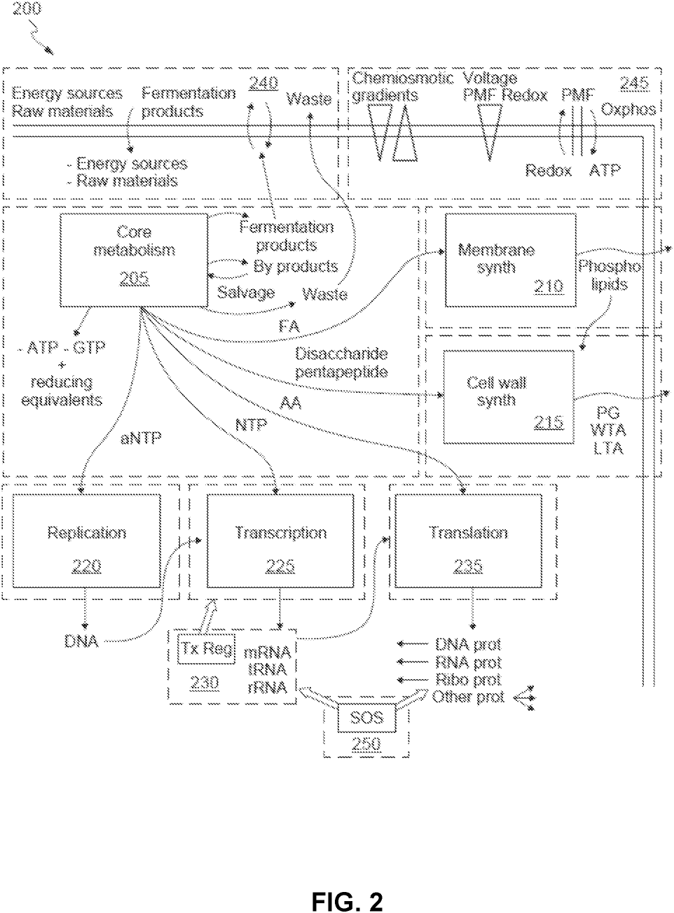

As another example, the interface may further or alternatively be configured to receive an input that corresponds to a selection of one or more default modules and a selection of a model type to be used for each of one or more modules. For example, the interface may include one or more modules (as shown in FIG. 2) representing distinct biological functions in a biological system model, and for each module: a name of the module, a default model type for the module and an option configured to receive a selection of another model type for the module (e.g., that identifies one or more other model types that can be selected for the module).

Default structure of a simulation (e.g., corresponding to default modules, default parameters, default initial-state values and/or default model selections) can be determined based on detected internal or external content and/or based on lab results (e.g., results from physical experiments). The content can include (for example) online, remote and/or local content that is collected by a content bot 115. Content bot 115 can (for example) include a crawler that performs a focused crawling and/or focused browsing (for example) the Internet, a part of the Internet, one or more pre-identified websites, a remote (e.g., cloud-based) storage system, a part of a remote storage system, a local storage system and/or a part of a local storage system. The crawling can be performed in accordance with one or more crawling policies and/or one or more queries that corresponds to one or more modules and/or models (e.g., where each query includes a variable name, representation or description and/or a cellular-function name, .representation or description).

The lab results can be received from a wet-lab value detection system 120, which can be configured to trigger performance of one or more investigations (e.g., physical experiments) to detect and/or measure data corresponding to an initial-state value and/or data corresponding to a characteristic or parameter of a biological system. Wet-lab value-detection system 120 can transmit one or more results of the investigation(s) back to simulation controller 105, which may thereafter determine and/or define a default initial-state value or parameter or a possible modification thereof based on the result(s).

Interaction system 100 further includes a simulation validator 125, which can be configured to validate performance of a simulation. The validation may be performed based on pre-identified indications as to how a biological system functions normally and/or given one or more perturbations. Such indications can be defined based on content collected from content bot 115 and/or results from wet-lab value-detection system 120. The data used to validate the simulation may include (for example) one or more balanced values, one or more values indicative of cell dynamics, one or more steady-state values, one or more intermediate values and/or one or more time-course statistics. Simulation validator 125 may return a performance result that includes (for example) a number, category, cluster or binary indicator to simulation controller 105. Simulation controller 105 may use the result to determine (for example) whether a given simulation configuration is suitable for use (e.g., in which case it may be selectable in an interface).

After a simulation is configured with definitions and/or selections of modules, module-specific models, parameters and/or initial-state values, simulation controller 105 can execute the simulation (e.g., in response to receiving an instruction from user device 110 to execute the simulation). The simulation execution can produce one or more simulation results, which may include (for example) one or more balanced values, kinetic values, etc. For example, the simulation can identify a solution for a set of reaction-corresponding stoichiometric equations using linear algebra, such that production and consumption of metabolites represented in the equations is balanced. Notably, this balance may be specific to a given module and need not be achieved for all metabolites produced or consumed by reactions for a given module (e.g., as a non-zero net production or consumption of one or more boundary metabolites may be predefined and/or a target result for a module). Simulation controller 105 can transmit the results (e.g., via an interface) to user device 110.

In some instances, the results can be used to trigger and/or define a subsequent experiment. For example, simulation controller 105 may determine whether a given predefined condition is satisfied based on the results and, if so, may transmit simulation-specific data (e.g., indicating one or more initial-state values, parameters, mutations corresponding to simulation definitions, etc.) to an experimental system 130. The transmission may be indicative of and/or include an instruction to perform an experiment that corresponds to the simulation.

As another example, upon receiving simulation results from simulation controller 105, user device 110 can present an interface that includes some or all of the results and an input component configured to receive input corresponding to an instruction to perform an experiment that corresponds to the simulation. Upon receiving a selection at the input component, user device 110 may transmit data corresponding to the simulation to experimental system 130. After performing a requested experiment, experimental system 130 may return one or more results to simulation controller 105 and/or user device 110.

FIG. 2 shows an illustrative representation of given biological system model 200. The overall modeling strategy includes partitioning the biological system model 200 into modules that can be modeled separately, using a methodology and level of detail appropriate to and/or selected for each module. The partitioning and level of detail for each module can be selected based on (for example) the experiments or simulations that are to be run by the model (e.g., the questions trying to be solved by the model). The selection may be made by the modeler and/or computing system (e.g., the interaction system 100 described with respect to FIG. 1). For example, a user working through an interface of an integrated development environment, a script, and/or an automated system may be implemented to select one or more modules and select a model type to be used for each of one or more modules to ultimately generate the biological system model 200. Additionally or alternatively, the partitioning can be customized and depend on an assessment of the biological functions defined for the initial high-level data set. For example, a separate module may be defined to represent each of the following biological functions: core metabolism 205, membrane synthesis 210, cell-wall synthesis 215, DNA replication 220, transcription 225, transcription regulation 230, translation 235, RNA salvage (not shown), protein and RNA maturation, protein salvage (not shown), transmembrane transport 240 (including electron chain, oxidative phosphorylation, redox, and pH interconversion activity 245), signal transduction (not shown), stress response and growth rate regulation 250, cell division, chemotaxis (not shown), and cell-cell signaling (not shown).

Biological system model 200 can include at least one module that handles core metabolism 205. One possible core metabolic module uses an FBA model, which takes its general shape from standalone FBA, but includes modifications that account for interactions of the core metabolic module with other modules. Each of one, more or all other modules may have their own production and consumption of some of the same molecules within the FBA network, as described in further detail herein. However, as should be understood to those of ordinary skill in the art, an FBA model does not have to be incorporated into the overall biological system model 200 in order for every simulation to work. Instead, various types of models can be used for the modules (e.g., core metabolism 205, membrane synthesis 210, cell-wall synthesis 215, etc.) so long as the type of models can be configured to read values from the state vector and return a list of changes that should be made to the state vector.

For one exemplary instantiation of biological system model 200, core metabolism 205, membrane synthesis 210, and cell-wall synthesis 215 may be encompassed as a single FBA problem, whereas DNA replication 220, transcription 225, transcription regulation 230, and translation 235 may be isolated from the rest of the metabolic network. Meanwhile, transcription 225 and translation 235 may use a template synthesis model, and DNA replication 220 may use a bulk mass-flow model. Transcription regulation 230 may be empirical and static. Optionally, RNA salvage may be modeled using constant non-specific degradation, polymerized DNA, RNA, and protein levels may be determined by the intrinsic rates of the processes that produce them, and the remainder of the components are provided as inputs or parameters of the model.

For another exemplary instantiation of biological system model 200, core metabolism 205 may be encompassed as a single FBA problem. The balance of internal metabolite pools and the supply of building blocks for other processes may be maintained by core metabolism 205. DNA replication 220, transcription 225, transcription regulation 230, and translation 235 may then be isolated from the rest of the metabolic network. Membrane biosynthesis 210 and cell-wall synthesis 215 may be modeled by substrate- and catalyst-driven kinetics. Import and export rates and all exchange with the environment may be driven by the kinetics of membrane transport. Transcription 225 and translation 235 may use a template synthesis model, and DNA replication 220 may use a bulk mass-flow model. Transcription regulation 230 may be empirical and static. Optionally, RNA salvage may be modeled using representations of constant non-specific degradation, while polymerized DNA, RNA, and protein levels may be determined by the intrinsic rates of the processes that produce them, and the remainder of the components for the biological system can be provided as inputs or parameters of the model.

For another exemplary instantiation of biological system model 200, core metabolism 205 may be encompassed as an FBA problem, whereas one or more of membrane synthesis 210, cell-wall synthesis 215, DNA replication 220, transcription 225, transcription regulation 230, and translation 235 can be isolated from the rest of the metabolic network. The balance of internal metabolite pools and the supply of building blocks for other processes may be maintained by core metabolism 205. Membrane biosynthesis 210 and cell-wall synthesis 215 may be modeled by substrate and catalyst driven kinetics. Import and export rates, and all exchange with the environment may be driven by the kinetics of membrane transport. Redox balance, pH, and chemiosmotic gradients may be maintained explicitly. DNA replication 220, transcription 225 and translation 235 may use models based on initiation, elongation, and termination, Transcription regulation 230 may be pattern driven. Stress response and growth rate regulation 250 may be modeled using feedback control mechanisms. Optionally, RNA salvage may be modeled using constant non-specific degradation, while polymerized DNA, RNA, and protein levels may be determined by the intrinsic rates of the processes that produce them, and the remainder of the components for the biological system can be provided as inputs or parameters of the model.

While the biological system model 200 has been described at some length and with some particularity with respect to several described modules, combinations of modules, and simulation techniques, it is not intended that the biological system model 200 be limited to any such particular module configuration or particular embodiment. Instead, it should be understood that the described embodiments are provided as examples of modules, combinations of modules, and simulation techniques, and the modules, combinations of modules, and simulation techniques are to be construed with the broadest sense to include variations of modules, combinations of modules, and simulation techniques listed above, as well as other modules, combinations of modules, and simulation techniques configurations that could be constructed using a methodology and level of detail appropriate to each module and the biological system model 200.

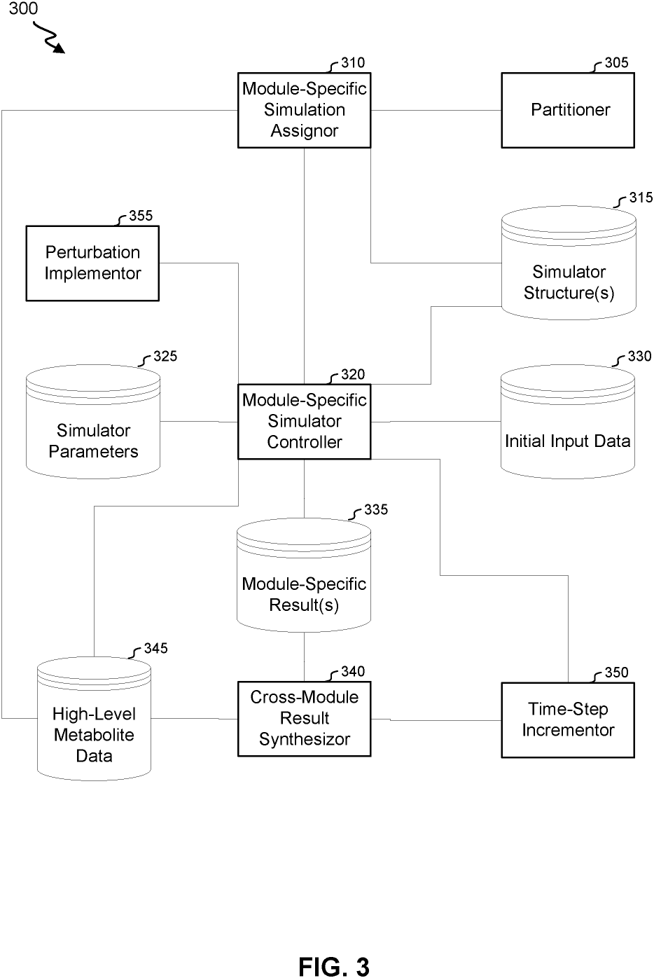

FIG. 3 shows a simulation controller 300 that dynamically integrates results generated by different types of models configured by an integrated development environment (e.g., the interaction system 100 described with respect to FIG. 1) to simulate higher-level states and reactions of a biological system model (e.g., biological system model 200 as described with respect to FIG. 2) according to various embodiments. A partitioner 305 that can identify one or more modules to potentially use for a simulation. In some instances, the modules are identified to correspond to distinct biological functions or physiological processes within a biological system model. Nonetheless, at least one module (e.g., a primary module) may address in more detail or cover a larger set of biological functions (e.g., correspond to a core level of physiology across the biological system such as general metabolism of the biological system), whereas at least one other module (e.g., a secondary module) may address in less detail or cover a smaller set biological function (e.g., correspond to transcription and/or translation).

A module-specific simulation assignor 310 may assign, to each module, a simulation type. The simulation type can be selected from amongst one or more types that are associated with the module and/or corresponding physiological process. The one or more types may differ with regard to (for example) a degree of detail to which a physiological process is modeled and/or how the process is modeled. For example, the one or more types may include a simulation using a metabolism-integrated model (e.g., in which specific end products are added to an objective function of a metabolism-based model), substrate- and/or catalyst-drive model using kinetic parameters and reactions, and/or higher-order structure model. A structure for each simulation type (e.g., that indicates how the simulation is to be performed and/or program code) is included in a simulator structure data store 315. Simulator structure data store 315 can further store an association between each simulation type and one or more modules for which the simulation type is associated and is permitted for selection for use.

A module-specific simulator controller 320 can identify, for each module, one or more simulation parameters and an input data set. The simulation parameters may be retrieved from a local data store (e.g., a simulator parameters data store 325) or from a remote source. Each of one or more of the simulation parameters may have been identified based on (for example) user input, a data-fitting technique and/or remote content. The parameter(s), once selected, may be fixed across time-step iterations.

At an initial time step, the input data set can include one or more initial input values, which may be retrieved from a local data store (e.g., an initial input data store 330) or from a remote source. Each of one or more of the initial input values may have been identified based on (for example) user input, a data-fitting technique and/or remote content. With respect to each subsequent time step, the input data set can include (for example) one or more results from a previous iteration of the module and/or one or more high-level results (e.g., cumulative or integrated results) generated from a previous iteration of the multi-module simulation. For example, a module-specific results data store 335 may store each of one, more or all results generated by the assigned simulation for each of one, more or all past time steps, and at least one of the stored results associated with a preceding time step (e.g., most recent time step) can be retrieved.

Upon identifying the input data set and parameters, module-specific simulator controller 320 can run the simulation assigned to the module. Execution of module-specific simulations may be performed concurrently, in parallel and/or using different resources (e.g., different processors, different memory and/or different devices). Results of the simulation run can be stored in module-specific results data store 335.

After results have been generated for each module, a cross-module result synthesizor 340 can access the module-specific results (from one or more module-specific results data stores or direct data availing) and synthesize the results to update high-level data such as a state vector (e.g., stored in a high-level metabolite data store 345). For example, a set of results generated by different modules but relating to a same variable may be identified. The results may be integrated by (for example) summing variable changes as indicated across the results (e.g., potentially with the implementation of one or more caps pertaining to a summed change or to a value of a variable after the summed change is effected). In some instances, a hierarchy is used, such that a result from one module (if available or if another condition is met) is to be exclusively used and a result from another module is to otherwise be used.

Upon synthesizing the results, a time-step incrementor 350 can increment a time step to a next time step so long as the simulation has not completed. It may be determined that the simulation is complete when (for example) processing for a predefined number of time steps has been performed, a particular result is detected (e.g., indicating that a target cell growth has occurred or that a cell has died) or steady state has been reached (e.g., as indicated by values for one or more predefined types of results differing by less than a predefined threshold amount across time steps). When the time step is incremented, module-specific simulator controller 320 can, for each module, collect a new input data set and run the assigned simulation. When the simulation is complete, an output can be generated to include one or more module-specific results, some or all high-level data and/or processed versions thereof. For example, the output may include time-course data for each of one or more metabolites, growth of the biological system over a time period (e.g., as identified by a ratio of availability values of one or more particular metabolites at a final time step as compared to availability values at an initial time step) and/or a growth rate. The output can be transmitted to another device (e.g., to be presented using a browser or other application) and/or presented locally.

Multi-module simulation controller 300 can also include a perturbation implementor 355. Perturbation implementor 355 can facilitate presentation of an interface on a user device. The interface can identify various types of perturbations (e.g., mutations). Perturbation implementor 355 may facilitate the presentation by transmitting data (e.g., HTTP data) to a user device, such that the interface can be presented online. Perturbation implementor 355 can detect a selection that corresponds to a particular perturbation and can send an indication to module-specific simulator controler 320. Module-specific simulator controller 320 can use functional gene data to determine how the mutation affects one or more metabolites and/or one or more simulated processes. A structure of a simulator, one or more simulator parameters and/or one or more initial-input values may then be adjusted in accordance was the perturbation’s effects. Thus, multi-module simulation controller 300 can generate output that is indicative of how the perturbation affects (for example) physiological processes and/or growth of the cellbiological system.

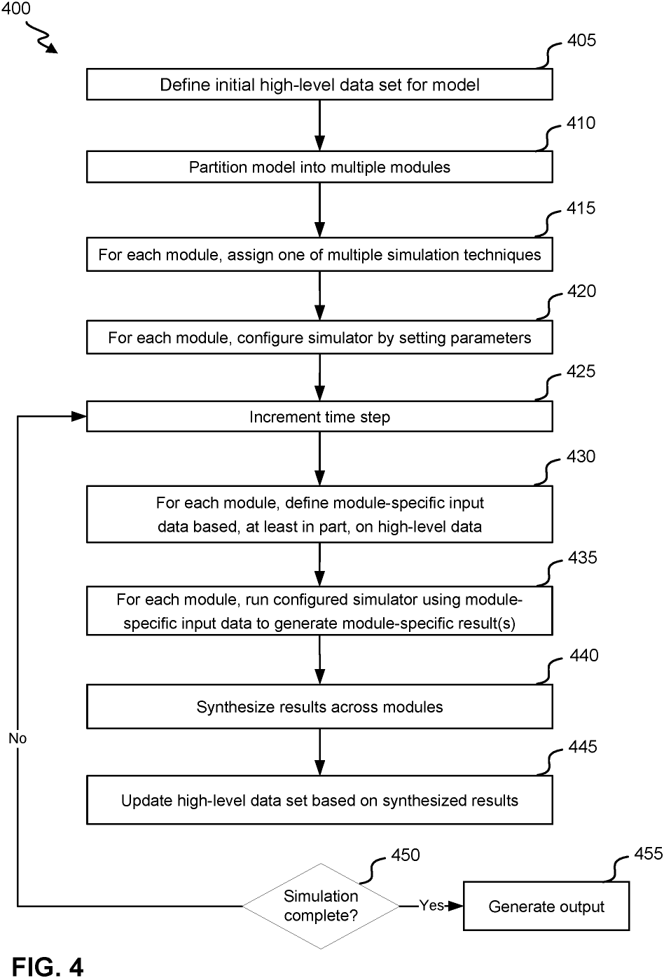

FIG. 4 shows a process 400 for dynamically synthesizing results generated by multiple simulators to simulate higher-level results according to various embodiments. In some embodiments, the processes depicted in process 400 are implemented by the interaction system 100 of FIG. 1, and discussed with respect to the simulation controller 300 of FIG. 4. Process 400 begins at block 405 at which an initial high-level data set is defined for a biological system model. The initial high-level data set can identify (for example) variables, which may be referred to as the state of the biological system model or the state of the simulation, and these variables may be structured as a data structure (e.g., a state vector) and updated throughout a simulation run. In some instances, the variables include an initial availability of each of a set of molecules such as metabolites. The initial availability may be defined based on (for example) a default value, user input, data extracted from content (e.g., online content, remote content or local content that pertains to the molecules), etc. In some instances, the initial availability is determined based on whether any perturbation was identified (e.g., via user input) for a given simulation. If a perturbation was identified, the initial availability may be determined based on a particular perturbation that was identified and by using (for example) a look-up table to determine for which molecule(s) the perturbation affects an availability value and characteristics of such effect.

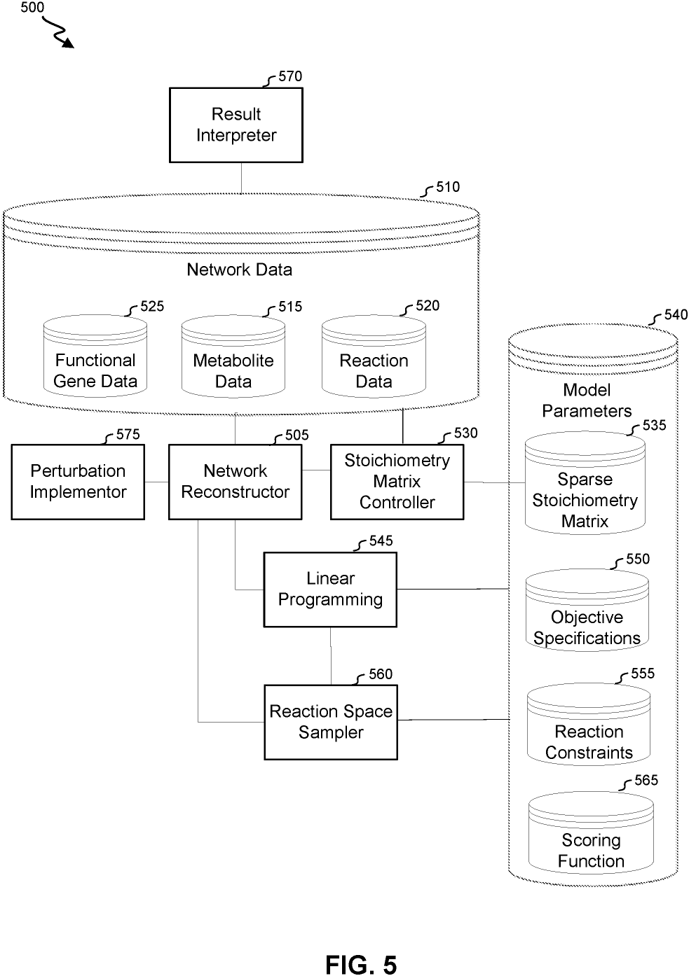

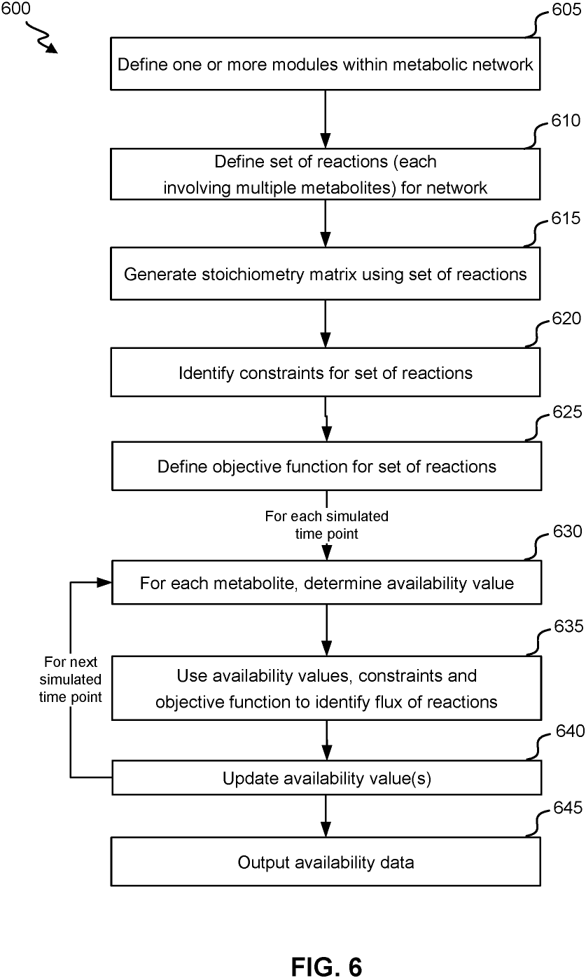

At block 410, a biological system model (e.g., a whole cell model) is partitioned into multiple modules. The partitioning can depend on metabolite dependencies and/or biological-functioning assessment. For example, a separate module may be defined to represent each of the following biological functions: core metabolism, membrane synthesis, cell-wall synthesis, DNA replication, transcription, transcription regulation, translation, RNA salvage, protein and RNA maturation, protein salvage, transmembrane transport (including electron chain, oxidative phosphorylation, redox, and pH interconversion activity), signal transduction, stress response and growth rate regulation (SOS), cell division, chemotaxis, and cell-cell signaling, as discussed in further detail with respect to FIG. 2. In some instances, two or more of these functions may be represented in a primary module that models cell composition and growth using a single model. Particular cellular functioning need not be explicitly modeled and instead dynamics of end products of the particular cellular functioning may be modeled. For example, a primary module may use a flux-based analysis or a simulation technique as described herein (e.g., in relation to FIG. 5 or FIG. 6).

In some instances, the partitioning may be performed based on user input and/or one or more default configurations. For example, an interface may be presented that identifies each potential separate module (e.g., an interface may be presented via simulation controller 105 as described with respect to FIG. 1). A default configuration may be to integrate the module into a primary module (e.g., a core metabolism module) unless a contrary input is received or to perform a simulation using modeling specific to the module unless a contrary input is received. For example, an interface may be configured to receive one or more selections of modules that are to be excluded from a primary module and to then integrate each other module into the primary module.

At block 415, for each module, one or more simulation techniques are assigned to the module. A simulation technique may include a model type. In some instances, a simulation technique that is assigned to a primary module includes a flux-based analysis or other simulation technique, as described herein. In some instances, a simulation technique includes a mechanistic model, a kinetic model, a partial kinetic model, a substrate- and/or catalyst-driven model, and/or a structural model. The simulation technique may be assigned based on (for example) user input and/or one or more predefined default selections. For example, for each secondary module, a default selection may be predefined that represents particular functioning of the module, and for each primary module, a default selection may be predefined that simulates dynamics of metabolites across a simulated time period. An interface may identify, for each module, the default selection along with one or more other simulation techniques that are associated with the module (e.g., with the association(s) being based on stored data and/or a predefined configuration). User input may then indicate that an alternative simulation technique is to be used for one or more modules.

At block 420, for each module, a simulator is configured by setting parameters and variables. The parameters (e.g., numeric values) may correspond to inputs to be used in the simulation technique assigned to the module and that are not changed across time steps of the simulation. The particular parameters may be determined based on (for example) stored data, content, a communication from another system and/or user input. The one or more module-specific or cross-module variables (e.g., identifying an initial availability of one or more metabolites) may correspond to inputs to be used in the simulation technique assigned to the module and may be changed across time steps of the simulation. For example, a parameter may be determined for a simulator that sets a minimum viable pH in the cytoplasm (below which the cell dies), and a variable may be identified that describes a current pH in the cytoplasm. The variable (current pH) might change throughout the simulation; however, the parameter (the minimum possible pH) would not change and remains fixed. An initial value of the pH variable may be identified, e.g., the value at the start of the simulation may be set in step 405 or if it is module specific then it may be set in step 420, and like the minimum pH parameter this would be used as an input into the simulation. The values of variables and parameters are both inputs, but the distinction is that variables can change from their initial values, and parameters are fixed throughout the simulation run.

At block 425, a time step is incremented, which can initially begin a given simulation. At block 430, for each module, module-specific input data is defined at least in part on the high-level data. More specifically, a high-level data structure may identify, for each of a set of molecules (e.g., metabolites), an availability value. Each availability value may initially be set to an initial availability value, which may thereafter be updated based on processing results from each module that relates to the molecule. For a given module, at each time step, a current availability value can be retrieved from the data structure for each molecule that pertains to the simulation technique assigned to the module. The module-specific input data may further include one or more lower-level values that are independent from processing of any other module. For example, one or more variables may only pertain to processing of a given module, such that the module-specific input data may further include an initial value or past output value that particularly and exclusively relates to the module.

At block 435, for each module, the configured simulator assigned to the module is run using the module-specific input data to generate one or more module-specific results. The one or more module-specific results may include (for example) one or more updated molecule availability values and/or a change in one or more availability values relative to corresponding values in the input data.

At block 440, results can be synthesized across modules. The synthesis may include summing differences across modules. For example, if a first module’s results indicate that an availability of a given molecule is to be increased by 5 units and a second module’s results indicate that an availability of the given metabolite is to be decreased by 3 units, a net change may be calculated as being an increase in 2 units. The net change can then be added to a corresponding availability value for the molecule that was used for the processing associated with the current time step and returned as a list of changes that should be made to the state vector. One or more limits may be applied to a change (e.g., to disallow changes across time steps that exceed a predefined threshold) and/or to a value (e.g., to disallow negative availability values and instead set the value to zero).

At block 445, the high-level data set is updated based on the synthesized results. The update can include adding data to a data structure such as a state vector from which one or more modules retrieve high-level data. The added data can include the synthesized results in association with an identifier of a current time step. Thus, the data structure can retain data indicating how an availability of a metabolite changed over time steps. It will be appreciated that alternatively the update can include replacing current high-level data with the synthesized data.

At block 450, it is determined whether the simulation is complete. The determination may be based on a number of time steps assessed, a degree to which data (e.g., high-level data) is changing across time steps, a determination as to whether a steady state has been reached, whether one or more simulated biological events (e.g., cell division or cell death) have been detected, etc. If the simulation is not complete, process 400 returns to block 425.

If the simulation is complete, process 400 continues to block 455, at which an output is generated. The output may include some or all of the high-level data and/or some or all of the module-specific results. For example, the output may include final availability values that correspond to a set of metabolites and/or a time course that indicates a change in the availability of each of one or more metabolites over the simulated time period. The output may be presented at a local device and/or transmitted to another device (e.g., for presentation).

FIG. 5 shows a module-specific simulation controller 200 to simulate states and reactions of modules configured by an integrated development environment (e.g., the interaction system 100 described with respect to FIG. 1) according to various embodiments. A network reconstructor 205 can be configured to use a model to simulate actions performed by a module of a biological system model (e.g., biological system model 200 as described with respect to FIG. 2). In some instances, the model is flux balance analysis, and/or the model is configured to solve for updated state values based on a set of equations that represent concentration changes in the network (e.g., a metabolic network). As should be understood to those of ordinary skill in the art, a biological system model such as a whole cell model does not have to include an FBA module. For example, from the framework described herein, biological processes such as core metabolism may be modeled that is completely different from FBA. In such an instance, part or all of the description and drawings pertaining to FIGS. 5 and 6 that is specific to FBA (e.g., objective functions, constraints, and linear programming) may not be relevant to that particular instantiation of the model or to simulations run with that model. However, many of the components and techniques described with respect to FIGS. 5 and 6 could be applied to simulate states and reactions of modules implemented by other models. For example, any module can read values from the state vector and return an indication of one or more changes that should be made to the state vector. The FBA module (if it’s even present in a particular instantiation of the model) may read and return more values than any other model, but a module modeled with FBA need not be handled by the simulation controller 300 any differently from other modules and/or models described herein.

Network reconstructor 505 can access a set of network data (e.g., parameters and variables) stored in a network data store 510 to define the model. Metabolite data 515 can identify each metabolite of a metabolome. As used herein, a “metabolite” is any substance that is a product of metabolic action or that is involved in a metabolic process including (for example) each compound input into a metabolic reaction, each compound produced by a metabolic reaction, each enzyme associated with a metabolic reaction, and each cofactor associated with a metabolic reaction. The metabolite data 515 may include for each metabolite (for example) one or more of the following: the name of the metabolite, a description, neutral formula, charged formula, charge, spatial compartment of the biological system and/or module of the model, and identifier such as PubChem ID. Further, metabolite data 515 can identify an initial state value (e.g., an initial concentration and/or number of discrete instances) for each metabolite.

Reaction data 520 can identify each reaction (e.g., each metabolic reaction) associated with the model. For example, a reaction can indicate that one or more first metabolites is transformed into one or more second metabolites. The reaction need not identify one-to-one relationships. For example, multiple metabolites may be defined as reaction inputs and/or multiple metabolites may be defined as reaction outputs. The reaction data 520 may include for each reaction (for example) one or more of the following: the name of the reaction, a reaction description, the reaction formula, a gene-reaction association, genes, proteins, spatial compartment of the biological system and/or module of the model, and reaction direction. Further, the reaction data 520 can identify, for each metabolite of the reaction, a quantity of the metabolite, which may reflect the relative input-output quantities of the involved metabolites. For example, a reaction may indicate that two first metabolites and one second metabolite are input into a reaction and that two third metabolites are outputs of the reaction. The reaction data 520 can further identify an enzyme and/or cofactor that is required for the reaction to occur.

Functional gene data 525 can identify genes and relationships between genes, proteins, and reactions, which combined provide a biochemically, genetically, and genomically structured knowledge base or matrix. Functional gene data 525 may include (for example) one or more of the following: chromosome sequence data, the location, length, direction and essentiality of each gene, genomic sequence data, the organization and promoter of transcription units, expression and degradation rate of each RNA transcript, the specific folding and maturation pathway of RNA and protein species, the subunit composition of each macromolecular complex, and the binding sites and footprint of DNA-binding proteins, Network reconstructor 505 can use the functional gene data 525 to generate or update one or more Gene-Protein-Reaction expressions (GPR), which associate reactions with specific genes that triggered the formation of one or more specific proteins. Typically a GPR takes the form (Gene A AND Gene B) to indicate that the products of genes A and B are protein sub-units that assemble to form a complete protein and therefore the absence of either would result in deletion of the reaction. On the other hand, if the GPR is (Gene A OR Gene B) it implies that the products of genes A and B are isozymes (i.e., each of two or more enzymes with identical function but different structure) and therefore absence of one may not result in deletion of the reaction. Therefore, it is possible to evaluate the effect of single or multiple gene deletions by evaluation of the GPR as a Boolean expression. If the GPR evaluates to false, the reaction is constrained to zero in the model. Thus, gene knockouts can be simulated in the model.

A stoichiometry matrix controller 530 can use reaction data 520 to generate a stoichiometry matrix. Along a first dimension of the matrix, different compounds (e.g., different metabolites) are represented. Along a second dimension of the matrix, different reactions are represented. Thus, a given cell within the matrix relates to a particular compound and a particular reaction. A value of that cell is set to 0 if the compound is not involved in the reaction, a postive value if the compound is one produced by the reaction and a negative value if the compound is one consumed by the reaction. The value itself corresponds to a cofficient of the reaction indicating a quantity of the compound that is produced or consumed relative to other compound consumption or production involved in the reaction.

Because frequently relatively few reactions correspond to a given compound, the stoichiometry matrix can be a sparse stoichiometry matrix 535. Sparse stoichiometry matrix 505 can be part of a set of model parameters (stored in a model-parameter data store 540) used to execute a module.

One or more modules may be configured to use linear programming 545 to identify a set of compound quantities that correspond to balancing fluxes identified in reactions represented in the stoichiometry matrix. Specifically, an equation can be defined whereby the product of the stoichiometry matrix and a vector representing a quantity for each of some of the compound quantities is set to zero. (It will be appreciated that the reactions may further include quantities for one or more boundary metabolites, for which production and consumption need not be balanced.) There are frequently multiple solutions to this problem. Therefore, an objective function is defined, and a particular solution that corresponds to a maximum or minimum objective function is selected as the solution. The objective function can be defined as the product between a transposed vector of objective weights and a vector representing the quantity for each compound. Notably, the transposed vector may have a length that is equal to the first dimension of sparse stoichiometry matrix 535, given that multiple reactions may relate to a same compound.

The objective weights may be determined based on objective specifications 550, which may (for example) identify one or more reaction-produced compounds that are to be maximized. For example, the objective weights can be of particular proportions of compounds that correspond to biomass, such that producing compounds having those proportions corresponds to supporting growth of the biological system.

Each reaction may (but need not) be associated with one or more of a set of reaction constraints 555. A reaction constraint may (for example) constrain a flux through the reaction and/or enforce limits on the quantity of one or more compounds consumed by the reaction and/or one or more compounds produced by the reaction.

In some instances, linear programming 545 uses the sparse stoichiometry matrix 535 and reaction constraints 550 to identify each solution that complies with the constraints. When multiple solutions are identified, objective specifications 550 can be used to select from amongst the potential solutions. However, in some instances, no solution is identified that complies with sparse stoichiometry matrix and reaction constraints 555 and/or the only solution that complies with the matrix and constraints is not to proceed with any reaction.

A solution can include one in which, for each of a set of metabolites, a consumption of the metabolite is equal to a production of the metabolite. That is not to say that this balance must be achieved for each metabolite, as a set of reactions involve one or more “boundary metabolites” for which this balance is not achieved. For example, glucose can be consumed at a given rate, and/or acetate can be produced at a given rate.

However, frequently coefficients from reaction data 520 are themselves estimates. If one or more reactions are inaccurate or if a reaction set is incomplete, using this type of linear-programming technique can result in outcomes indicating that the biological system failed to grow merely due to a misestimate in a reaction. Thus, another technique is to permit growth (as a result of simulated occurrence of multiple reactions) even when no balanced solution is identified.

For example, a reaction space can be defined based on sparse stoichiometry matrix 535 and reaction constraints 555. The space may have as many dimensions as there are reactions. Each dimension can be restricted to include only integer values that extend along a range constrained by any applicable constraint in reaction constraints 555. A reaction space sampler 560 can then determine, for each of some or all of the points within the reaction space, a cumulative quantity of each metabolite that would be produced based on the associated reactions. Reaction space sampler 560 can compare these quantities to those in the objective vector (e.g., by determining an extent to which proportions of compounds are consistent).

In these instances, a scoring function 565 can indicate how to score each comparison. For example if proportions of each of two potential solutions differ from the objective proportions by 2, but one potential solution differs by 2 for a single compound and another by 1 for each of two compounds, scoring function 565 can be configured to differentially score these instances. For example, different weights may be applied to different compounds, such that differences that affect a first compound are more heavily penalized than differences that affect a second compound. As another example, scoring function 565 may indicate whether a score is to be calculated by (for example) summing all compound-specific (e.g., weighted) differences, summing an absolute value of all compound-specific (e.g., weighted) differences, summing a square of all compound-specific (e.g., weighted) differences, etc. Reaction space sampler 560 can then identify a solution as corresponding to reaction coefficients that are associated with a highest score across the reaction space.

Network reconstructor 505 can receive results from each of linear programming 545 and/or reaction space sample 560. In some instances, linear programming 545 can further avail its results to reaction space sample 560. When a balanced solution is identified by linear programming 545, reaction space sampler 560 need not sample the reaction space and need not avail reaction-space results to network reconstructor 505.

Network reconstructor 505 can identify a solution as corresponding to one identified by linear programming 545 when a balanced solution is identified and as a highest-score potential solution identified by reaction space sampler 560 otherwise. The solution can then indicate the compounds produced by and consumed by the reactions performed in accordance with the solution-indicated flux. Network reconstructor 505 can update metabolite data 515 based on this production and consumption.

In some instances, a solution is identified for each of a set of time points rather than only identifying one final solution. The iterative time-based approach may be useful when module-specific simulation controller 500 is but one of a set of simulation controllers and metabolite data 515 is influenced by the performance of other modules. For example, metabolite data 515 may be shared across modules or may be defined to be a copy of at least part of a cross-module metabolite data set at each time point. The updates to the metabolites performed by network reconstructor 505 may then be one of multiple updates. For example, an update by network reconstructor 505 may indicate that a quantity of a specific metabolite is to increase by four, while a result from another module indicates that a quantity of the specific metabolite is to decrease by two. Then the metabolite may change by a net of +2 for the next time iteration.

A results interpreter 570 can generate one or more results based on the updated metabolite data 515. For example, a result may characterize a degree of growth between an initial state and a steady state or final time point. The degree of growth may be determined based on a ratio between values of one or more metabolites at a current or final time point relative to corresponding values at an initial (or previous) time point. The one or more metabolites may correspond to (for example) those identified in an objective function as corresponding to biomass growth. As another example, a result may characterize a time course of growth. For example, a result may identify a time required for metabolite changes that correspond to a representation of a double in growth or a time constant determined based on a fit to values of one or more time series of metabolite values. The result(s) may be output (e.g., locally presented or transmitted to a remote device, such as a user device). The output can facilitate a presentation of an interface that indicates one or more simulation characteristics (e.g., one or more default values in terms of initial-state values or reaction data and/or one or more effected perturbations).

Module-specific simulation controller 500 can include a perturbation implementor 575. Perturbation implementor 575 can facilitate presentation of an interface on a user device. The interface can identify various types of perturbations. For example, each perturbation may correspond to a particular type of genetic mutation. Perturbation implementor 575 may facilitate the presentation by transmitting data (e.g., HTTP data) to a user device, such that the interface can be presented online. Perturbation implementor 575 can detect a selection that corresponds to a particular perturbation (by receiving a communication indicative of the selection from the user device) and can send an indication to network reconstructor 505 that the perturbation is to be effected. Network reconstructor 505 can use functional gene data 525 to determine how the mutation affects one or more metabolites, which can affect whether and/or how one or more reactions can occur. Stoichiometry matrix controller 530 can then generate a perturbed sparse stoichiometry matix 535 representing the perturbed state corresponding to the genetic mutation, and a solution can be identified as previously indicated based on the perturbed sparse stoichiometry matrix.

FIG. 6 shows a process 600 for using a simulator to generate metabolite time-course data according to various embodiments. In some embodiments, the processes depicted in process 600 are implemented by the interaction system 100 of FIG. 1, and discussed with respect to the module-specific simulation controller 500 of FIG. 5. Process 600 begins at block 605, at which a one or more modules within a metabolic network (e.g., of a biological system) are defined. The module(s) can be defined based on which parts of the network exhibit relative functional independence and/or correspond to substantial independence in terms of biological activity. In some instances, a default is to define one or more particular modules that are to be simulated using a model type that differs from a default model type (e.g., flux-based analysis) and to define the rest of the network as a single module using a default model type.

At block 610, a set of reactions is defined for the network. In some instances, the set of reactions are defined for the module (or each module) that corresponds to the default model type. The set of reactions can indicate how various molecules such as metabolites are consumed and produced through part of all of a life cycle of a biological system. Each reaction thus identifies one or more metabolites that are consumed, one or more metabolites that are produced and, for each consumed and produced metabolite, a coefficient (which may be set to equal one) indicating a relative amount that is consumed or produced. The reaction may further include an identification of one or more enzymes, one or my cofactors and/or one or more environmental characteristics that are required for the reaction to occur and/or that otherwise affects a probability of the reaction occurring or a property of the reaction. The reactions may be identified based on (for example) online or local digital content (e.g., from one or more scientific papers or databases) and/or results from one or more wet-lab experiments.

At block 615, a stoichiometry matrix is generated using the set of reactions. Each matrix cell within the matrix can correspond to a particular metabolite and a particular reaction. The value of the cell may reflect a coefficient of the particular metabolite within the particular reaction (as indicated in the reaction) and may be set to zero if it is not involved in the reaction. In some instances, metadata is further generated that indicates, for each of one or more reactions, any enzyme, co-factor and/or environmental condition required for the reaction to occur.

At block 620, one or more constraints are identified for the set of reactions. In some instances, identifying the constraints may include identifying values for one or more parameters. For example, for each of one or more or all of the set of reactions, a constraint may include a flux lower bound and/or a flux upper bound to limit a flux, a quantity of a consumed or produced metabolite, a kinetic constant, a rate of production or decay of a component such as RNA transcript, an enzyme concentration or activity, a compartment size, and/or a concentration of an external metabolite. The constraint(s) may be identified based on (for example) user input, online or local data, one or more communications from a wet-lab system, and/or learned from statistical inference.

At block 625, an objective function is defined for the set of reactions. The objective function may identify what is to be maximized and/or what is to be minimized while identifying a solution. The objective function may (for example) identify a metabolite that is produced by one or more reactions or a combination of metabolites that is produced by one or more reactions. The combination may identify proportions of the metabolites. However, the objective function can have a number of limitations and may fail to reflect supply and demand within the other modules. Thus, in some instances, a limited objective function can be constructed to include a set of target values for each molecule within the metabolic network. The target values can incorporate intrinsic-rate parameters, supply rates of molecules, the consumption rates of molecules, and the molecule concentrations into a measurement of target concentrations of the molecule given supply, demand, and an “on-hand” concentration of each molecule, which represents the concentration of a molecule immediately available to a reaction pathway. The target values may be calculated and incorporated into the objective function to produce the limited objective function. This may be in the form of calculating an absolute difference between the target value and the proportional flux contribution of each molecule. This may be in the form of scaling the proportional flux contribution of each molecule. This may be in the form of adding to the proportional flux contribution of each molecule. Any other mathematical modification of the proportional flux contribution of each molecule that adjusts this value by the target value may be used. The target values may be positive or negative. For purposes of unit conversion, so that target values can be included in the objective function and compared to the flux values, the target values may be constructed as rates.

Blocks 630-640 are then repeated for each of a set of simulated time points. At block 530, for each metabolite related to the set of reactions, an availability value is determined. For an initial value, the value may be identified based on (for example) user input, digital content and/or communication from another system. Subsequent values may be retrieved from a local or remote data object that maintains centralized availability values for the set of metabolites.

At block 635, the availability values, constraints and objective function are used to determine the flux of one, more or all of the set of reactions. The flux(es) may indicate a number of times that each of one, more or all of the reactions were performed in a simulation in accordance with the availability values, constraints and objective function. The flux(es) may be determined based on a flux-balance-analysis model. In some instances, the flux(es) may be determined based on a sampling of all or part of an input space representing different flux combinations and scoring each input-space using a scoring function.

At block 640, a centralized availability value of one or more metabolites is updated based on the determined flux(es). More specifically, for each metabolite, a cumulative change in the metabolite’s availability may be identified based on the cumulative consumption and cumulative production of the metabolite across the flux-adjusted set of reactions. The centralized availability value of the metabolite can then be incremented and/or decremented accordingly.

In some instances, at least one the one or more modules defined at block 505 are to be associated with a model that does not depend on (for example) a stoichiometry matrix and/or flux based analysis and/or that is based on physiological modeling. One or more modules based on one or more different types of models can also, at each time point, identify a change in metabolite availability values, and such changes can also be used to update a local or remote data object with centralized availability values. With respect to each metabolite, updates in availability values may be summed to identify a total change and/or updated availability value. In some instances, limits are set with respect to a maximum change that may be effected across subsequent time steps and/or a maximum or minimum availability value for a metabolite.

Blocks 630-640 may be repeated for each of multiple simulated time points in a simulation. In some instances, a predefined number of simulated time points are to be evaluated and/or simulated time points corresponding to a predefined cumulative time-elapsing period are to be evaluated. In some instances, a subsequent simulated time point is to be evaluated until a predefined condition is satisfied. For example, a predefined condition may indicate that metabolite values for a current simulated time point are the same or substantially similar as compared to a preceding simulated time point or a preceding simulated time period.

When process 600 returns to block 630 for evaluation of a next simulated time point, it will be appreciated that an availability value determined for a given metabolite need not be equal to the corresponding updated availability value from the previous iteration of block 540 and/or the sum of the previously determined availability value adjusted by the identified flux pertaining to the metabolite. Rather, a processing of the previous time point with respect one or more other modules may have also resulted in a change in the metabolite availability. Thus, the availability value for a given metabolite determined at block 530 for a current time point may be equal to the availability value determined at block 530 for a preceding time point plus the cumulative updates to the availability value across modules, with any limits imposed.

When all time points have been evaluated, process 600 proceeds to block 645 at which availability data is output. For example, the availability data may include, for each of one, more or all metabolites: an availability value (e.g., a final availability value) and/or a time course of the availability value. In some instances, the availability data is output with reference availability data. For example, when part or all of the processing performed to calculate the availability values was associated with a perturbation, the reference availability data may be associated with an unperturbed state. In some instances, a processed version of the availability data is output. For example, a comparison of availability values for particular metabolites across time points may be used to generate one or more growth metrics (e.g., a growth magnitude or rate), which may be output. Outputting the availability data can include (for example) locally presenting the availability data and/or transmitting the availability data to another device.

### Parameter and State Initialization for Model Training

As explained above, one approach for modeling is to partition a model into modules and to identify a simulation technique and parameters for each module. (See blocks 410-420 of process 400). It will be appreciated that a model may alternatively include a single module or may lack modules all together. For any of various types of model configurations (e.g., a model that includes multiple modules, a single module, or no modules), values for model parameters and initial state variables may be derived from one or more sources. For example, a model parameter or initial state variable may be defined to be a default value that was identified (e.g., for the parameter or state variable) at a time when the model was being constructed and/or before fitting the model to data . As another example, a model parameter or initial state variable may be a “learnable” value, that is obtained by fitting the model to data (also called a training period) . As yet another example, a model parameter or initial state variable may be assigned a value defined in a condition-specific assignment, where the condition corresponds to a particular simulated experimental condition.

In some embodiments, parameters (and/or states) for a model and/or one or more conditions (e.g., modules in a model) are learned during a training phase using a loss function. Training a machine-learning model can include optimization-based point estimation of parameters and sampling-based estimation of parameter distributions. The estimation of parameters and/or the estimation of parameter distributions can be performed using experimental data collected under diverse conditions.

However, defining parameters (and/or state initializations) for each of multiple modules can be a time- and resource-intensive effort that can require a large amount of training data. Further, separate condition-specific training may result in a situation where a value learned for a given parameter in one context or condition is different than a value learned for the parameter in another context or condition. This inconsistency may result in data abnormalities and erroneous results when a simulation involves a transition or interaction between the two contexts or between the two conditions. Additionally, the inconsistency may make it difficult to interpret what the model has learned and/or what factors are contributing to a model output.

In some embodiments, a new technique for learning model parameters is provided where each parameter is classified to indicate over which conditions the parameter is to have the same value. For example, a parameter may be classified as a “global” parameter that is to have the same value across all conditions, a “shared” parameter that is to have the same value across at least two conditions (but need not have the same value for one or more other conditions), or a “local” parameter where a condition-specific value for the parameter is learned for each condition.

A condition can correspond to a a use case for a model. For example, a model may be generated to predict a size or growth of a cell, and the model may be used to simulate a first condition in which the cell is in a first growth media and separately to simulate a second condition in which the cell is in a second growth media. Thus, the architecture of the model may (but need not) be the same in the two conditions, but each condition may be defined by a corresponding set of parameter values. Thus, the values of the particular set of parameters are configured to be unique across conditions. Such uniqueness relates to the combination of values and does not necessarily apply that each parameter value corresponding to a first condition is different than the corresponding values in all other conditions. In some instances, different conditions may correspond to differences as to (for example) whether one or more genetic knockouts is simulated, an expression level of one or more genes, a level of a reagent, and/or whether an enzyme is present in a simulation.

It will be appreciated that when there are shared and/or global parameters, fewer unique parameters need to be learned as compared to parameters that are used across conditions in the model (e.g., if an alternative baseline is to learn each parameter independently condition). Thus, when a shared or global parameter applies to multiple conditions, a first data structure may identify the single parameter as being learnable, and one or more other data structures may indicate that the parameter in each of the multiple conditions is to be a copy of the learned parameter. Alternatively or additionally, one or more parameter data structures can be generated that indicates that the parameter in a given condition is learnable and the parameter in the remaining conditions of the multiple conditions are to be copies of the parameter in the given condition.

Shared or global parameters can result in training efficiencies, such that the computational resources committed to training, the time for training, and a minimum size of a training data set are reduced as compared to treating all parameters as local parameters. Further, the interpretability of the model can be improved, and transitions between conditions can be less abrupt.

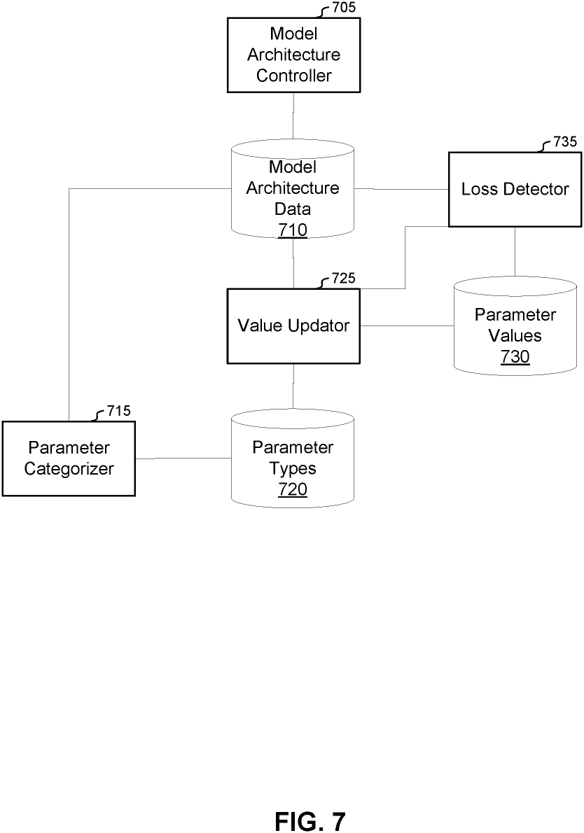

FIG. 7 shows a box diagram of an exemplary computing system for building a machine learning model. A model architecture controller 705 is configured to define model architecture data 710 for a machine-learning model. The machine-learning model can be configured such that all experimental conditions of interest can be simulated by the single model with appropriate parameter and initial state settings. For example, if there are two enzymes E1 and E2 used in separate experiments, the model could contain representations of both enzymes (and associated related reactions), and the activity of each enzyme could be set to zero in experiments where that enzyme is not present.

For each of the identified conditions, model architecture data 710 can identify an architecture that applies to the condition and a set of parameters that are to be used when the condition is satisfied. Model architecture data 710 can indicate how each of the parameters is to be integrated within a corresponding model architecture to influence how data received by a model is transformed into an output. In some instances, a single model architecture and/or one or more parameters are associated with multiple conditions.

A parameter categorizer 715 identifies a parameter type for each parameter in model architecture data 710. The parameter types can be stored in a parameter type data store 720. Each parameter may have a default value or condition-specific assignment that may be overridden as a result of training. As further described below, a parameter type may distinguish between global, shared, and local learnable parameters. Before a training stage begins, a value updator 725 assigns an initial value to each parameter and store the parameter values in a parameter value data store 730.

### Model Parameter Default Values

Default values specified at model construction time can be parameters and state values defined for model (e.g., specified in an SBML file from which the model is constructed). The default values may include values that were not learned. The default values may be specified separately from the model itself, e.g. as base_assignments in a configuration file. A characteristic of a model default value is that it is assigned once at the outset of a training run, and is then fixed unless overridden by assignments from the next two levels. Thus, for each default parameter, value updator 725 may identify a default value by (for example) retrieving the value from local or remote storage, detecting a user input that identifies the value, or requesting and receiving the value from an external data source.

### Defining Learnable Parameters - Global, Local and Shared Parameters

A learnable parameter is any value which is obtained from a proposed parameter vector during optimization or sampling. Learnable parameters can include model parameters and/or initial values for model state variables (e.g., enzyme concentrations). Exemplary model parameters include: a rate constant for a particular enzyme metabolizing a substrate into a product molecule (K_{cat}) and/or a reaction rate when a particular enzyme is fully saturated by a substrate (Vmax).

For each learnable parameter, value updator 715 can initialize the parameter with an initial value before training begins. The initial values may be identified by (for example) randomly or pseudorandomly selecting a value from a distribution or retrieving an initialization value from storage.

A learnable parameter can be a global parameter, a local parameter or a shared parameter.

When a parameter is a global parameter, parameter categorizer 715 configures the model such that a single value for the parameter (e.g., the value provided in the learnable parameter vector) is to be used to define the parameter across all conditions being simulated. Similarly, when an initial state is a global initial state, the model is configured such that a single value for the initial state (e.g., a value provided in the learnable parameter vector or in an initial-state vector) is to be used as the initial state across all conditions being simulated.

When a parameter is a local parameter, parameter categorizer 715 configures the model such that a value for the parameter is selected based on in which condition is being simulated at that time (or into which condition a simulation is transitioning). Thus, a local parameter with n conditions expands to n entries on the learnable parameter vector, each mapped to the same model parameter or initial state in one of the n conditions.

When a parameter is a shared parameter, parameter categorizer 715 configures the model such that a single value provided in the learnable parameter vector is applied to the model parameter or initial state across a set of conditions specified in the description of the parameter.

Learnable parameters can have one or more of the following attributes:
Parameter type: global, local, shared.
Name: name of the learnable parameter, which may be distinct from the name of the underlying model parameter or state variable.
Variable type: whether the learnable parameter maps to a model parameter or state variable.
Model name: name of the model parameter or state variable to which the learnable parameter will be mapped.
Conditions: for shared parameters, a set of condition names for which the learnable parameter value provided will be applied to the model.
Parameter Priors: for Bayesian inference or MAP estimation, a prior to be used in the estimation procedure, incorporating information into the modeling procedure.

Thus, parameter categorizer 715 can configure each learnable parameter 715 to specify how values in an optimized or sampled parameter vector are mapped onto model parameters or initial values for state variables. A learnable parameter may be defined by one or more of:
A name of the learnable parameter, which may be distinct from the model name;
An indication of any condition to which the parameter applies;
A model variable (parameter or state vector entry) to which the learnable parameter will be mapped (e.g., TargetVariable variable = 2);
The type of learnable parameter, containing any additional type-specific fields (e.g., an identification of one of: a global parameter, local parameter, or shared parameter);
If the parameter is not a global parameter (or if the parameter is a local or shared parameter), the parameter’s definition may further include two or more condition possibilities (each of which may include one or more conditions), where a different value may then be learned for each condition possibility;
A prior distribution (e.g., to support Bayesian inference).

It will be appreciated that an initial state can similarly be a global, local or shared initial state. Similarly, a learnable initial state may have one, more or all of the above-listed attributes of a learnable parameter. A learnable initial state may alternatively or additionally be defined by one, more or all of the above-listed definition elements of a learnable parameter. It will be appreciated that disclosures herein that relate to a parameter, parameter value, and/or a parameter vector may alternatively be applied to an initial state, initial-state value, and/or an initial-state vector. In some instances, a parameter vector includes one or more parameter values and one or more initial-state values.

Examples of local or shared parameters include:
Vₘₐₓ, which can be set to 0 in one or more conditions to simulate a knockout;
An external concentration of a reagent, which can be set to a non-zero value in one or more conditions to simulate adding the reagent to a medium;

An exemplary definition of a condition includes one or more variable-assignment techniques. Each variable-assignment technique can include an indication as to how a value is to be assigned to a parameter. In some instances, the one or more variable assignment techniques are organized in a hierarchy. For example, a first variable-assignment technique may indicate that a given parameter of a model is a global parameter that corresponds to enzymatic activity of a particular protein, and ten experimental conditions may be defined for the model. In one of those conditions, the gene the codes for the protein may have been deleted. Accordingly, the given parameter may be defined as a single global parameter for all conditions, though a second variable assignment protocol may be defined for the model to indicate that a value of “0” is to be defined for the given parameter in the one of the conditions. For example, in an illustrative instance, a global parameter corresponds to the enzymatic activity of a particular protein and there is data from 10 different experimental conditions. In one of those conditions, (e.g., condition 3) the gene that codes for that protein is deleted. A single global parameter may be defined for all conditions, but, in the condition 3, a value of 0 can be assigned to that parameter, since the enzyme is no longer present. Thus, the assigned value can overwrite the value of the global parameter.

### Learning Learnable Parameters - Global, Local and Shared Parameters

A loss detector 735 can use training data sets to calculate a loss function based on predicted results (corresponding to various values for learnable parameters) and true results. A result may include (for example) a next state of a simulation, a label, one or more next-time-step levels of a simulated reagent or product, etc. Value updator 725 can then update parameter values based on the loss function.

The predicted results can be generated by transforming corresponding inputs (e.g., representing a state of a simulation) using a parameter matrix. The parameter matrix may initially include default parameters values corresponding to each condition being simulated. In some instances, a predicted result is generated by identifying a current condition being simulated, retrieving - from the parameter matrix - a single row or column that corresponds to the condition (i.e., a condition-specific parameter vector), and using the retrieved values to generate the predicted result. A single condition-specific parameter vector corresponds to a single condition.

Once training begins, parameter values may be learned. For example, an optimization algorithm (e.g., gradient descent, batch gradient descent, stochastic gradient descent, or mini-batch gradient descent) can be used to identify updated values for parameters that are associated with a lower or minimal loss as identified by the loss function. A pre-identified learning rate can control the extent to which parameter values are allowed to be adjusted from current values and/or the extent to which parameter values are to be changed to correspond to a minimum loss.

### Loss Function

Value updator 725 uses the default parameters from the model to construct a parameter matrix P, which may be of a size n_conditions x n_parameters. At initialization, each row can be a copy of the model’s default parameter vector.

The model parameters can be mapped from the parameter vector V into the parameter matrix P. This mapping can include:
For a global parameter, the same value from a parameter vector V is copied into the full column (corresponding to different conditions) of the parameter matrix P corresponding to the model parameter that is being learned.
For a local parameter, the value in V for each condition that is copied to the entry of the mapping matrix P corresponding to the specified (condition, parameter) pair.
For a shared parameter, the value in V that is copied into an incomplete subset of entries in a column of the parameter matrix P, corresponding to the appropriate model parameter and condition subset.

Thus, it will be appreciated that there may be more values in the parameter matrix P than there are unique parameter values to be learned and/or represented in a loss-function space. Similarly, it will be appreciated that there may be more cumulative parameters (across conditions) used to generate predicted values (e.g., during training or afterwards) than there are unique parameter values to be learned and/or represented in a loss-function space.

For example, Table 1 shows an initial parameter matrix P for an exemplary instances where a model has three parameters X, Y, and Z and is to simulate three conditions A, B, and C. The default parameter values are X = 1.0, Y = 2.0, Z = 3.0.

TABLE 1

Conditions

| | | A | B | C |
|---|---|---|---|---|
| Parameter s | X | 1.0 | 1.0 | 1.0 |
| | Y | 2.0 | 2.0 | 2.0 |
| | Z | 3.0 | 3.0 | 3.0 |

In this illustration, parameter categorizer 715 identified Y and Z as learnable parameters, with Y being a global parameter and Z being a shared parameter across conditions A and B for parameter Z. The associated parameter-type information (stored in parameter type data store 720) may include:
param_y = LearnableParameter(
name=‘Y_global’,
variable=TargetVariable(model_parameter=‘Y’),
global_param=GlobalParameter())
param_z = LearnableParameter(
name=‘Z_shared’,
variable=TargetVariable(model_parameter=‘Z’),
shared_param=SharedParameter(conditions=[‘A’, ‘B’]))

For a particular invocation, a learnable parameter vector containing two values: {‘Y_global’: 2.5, ‘Z_shared_A_B′: 4.0} can be generated. Applying these values to the parameter matrix P results in an updated parameter matrix shown in Table 2.

TABLE 2

Conditions

| | | A | B | C |
|---|---|---|---|---|
| Parameter s | X | 1.0 | 1.0 | 1.0 |
| | Y | 2.5 | 2.5 | 2.5 |
| | Z | 4.0 | 4.0 | 3.0 |

In some instances, a condition is not associated with any condition-specific assignments. For example, Condition A may correspond to a “wild-type” condition. In some instances, a condition corresponds to a circumstance where a particular type of activity represented by a given parameter does not exist. For example, Condition B may correspond to an instance where an enzyme having activity represented by Parameter Y is knocked out, and Condition C may correspond to an instance where an enzyme having activity represented by Parameter Z is knocked out. The conditions may be specified as:
cond_b = [VariableAssignment(variable=TargetVariable(model_parameter=‘Y’), value=0.0)]
cond_c = [VariableAssignment(variable=TargetVariable(model_parameter=‘Z’), value=0.0)]

When value updator 725 applies these conditions to the parameter matrix P shown in Table 2, the result is shown in Table 3:

TABLE 3

Conditions

| | | A | B | C |
|---|---|---|---|---|
| Parameter s | X | 1.0 | 1.0 | 1.0 |
| | Y | 2.5 | 0 | 2.5 |
| | Z | 4.0 | 4.0 | 0 |

A model-execution controller (not shown) can then use the machine-learning model (having the architecture as indicated in model architecture data 710) and - upon detecting a given condition - configure the model with the parameter values associated with the corresponding column from the parameter matrix P shown in Table 3.

### Exemplary Process for Training and Using Machine Learning Model

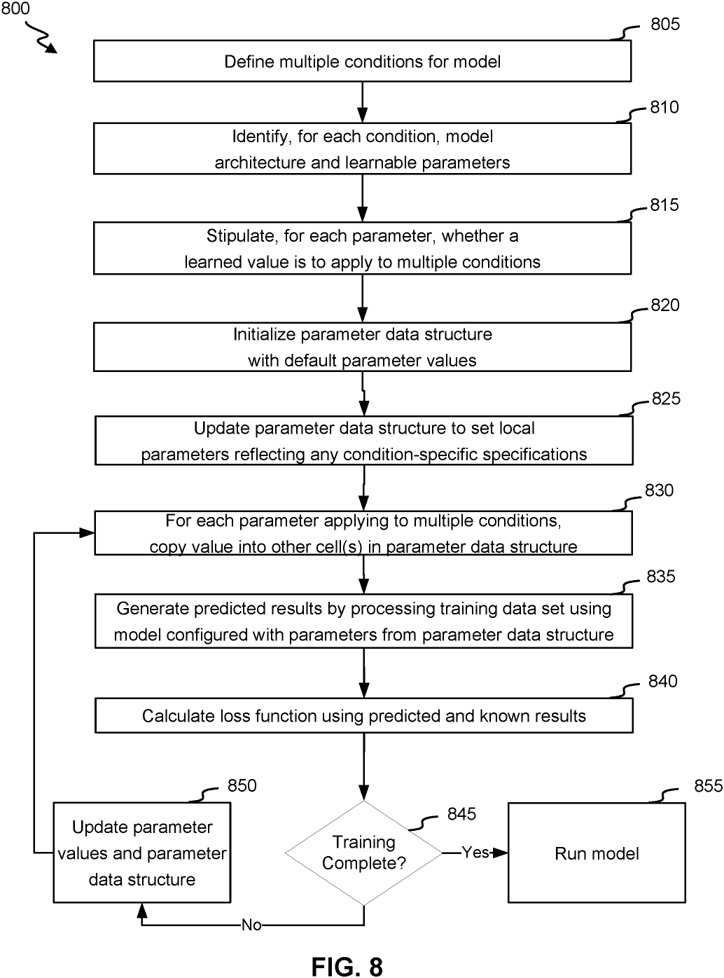

FIG. 8 shows a flowchart of an exemplary process 800 for training and using a machine learning model for use across different conditions. Process 800 begins at block 805 where model architecture controller 705 defines multiple conditions for a machine-learning model. Differences across the conditions may relate to (for example) whether one or more genetic knockouts is simulated, an expression level of one or more genes, whether an enzyme is present in a simulation, etc.

At block 810, model architecture controller 705 identifies, for each condition, a model architecture and a set of learnable parameters. For example, block 810 may include identifying -for each condition - a type of model (e.g., a flux-based analysis model, a mechanistic model, a kinetic model, a partial kinetic model, a substrate- and/or catalyst-driven model, etc.) and the learnable parameters that correspond to the model.

At block 815, parameter categorizer 715 stipulates, for each learnable parameter, whether a learned value is to apply to multiple conditions. If a given learnable parameter is to apply to multiple conditions, then the value for the parameter may be learned using a cumulative training data set across the multiple conditions, and the same value for the parameter can then be used in the condition-specific models. Determining whether a given learnable parameter is to apply to multiple conditions can involve (for example) determining or predicting an extent to which multiple parameters represent a same physiological consideration and/or predicting whether a given measurement is likely to exhibit different dependencies in different contexts. This can make it substantially easier to run the same model on the same same data set with different learnable parameter configurations, without changing other details (or even anything except the learnable parameter settings. By changing the learnable parameter settings, we can automatically increase or decrease model complexity in a principled manner.

At block 820, value updator 725 initializes a parameter data structure with default parameter values. Each default parameter value may be identified by (for example) selecting a value from a distribution (e.g., randomly, corresponding to a mode value, etc.). For each shared or global parameter, a single default parameter value can be identified and represented in the parameter data structure for each condition-specific parameter associated with the shared or global parameter.

At block 825, value updator 725 updates the parameter data structure to set local parameters reflecting any condition-specific specifications. The update may include setting a particular parameter value to 0, to a predefined constant, or to a maximum value. For example, a value of a parameter corresponding to a peptide concentration to be set to zero for a condition associated with a knock-out that prevents formation of the peptide. As another example, a value of an environmental condition can be set to a predefined constant for a condition associated with chemostat.

At block 830, for each parameter for which a learned value is to apply to multiple conditions, value updator 725 copies the value of the learned parameter into each other cell(s) corresponding to each (other) of the multiple conditions. During an initial run of block 830, the value that is copied can include a default value.

At block 835, a model-execution controller generates predicts results by processing at least part of training data set using the model (that includes the architecture and parameters corresponding to each condition) configured with parameter values from the parameter data structure. The execution may include determining a time-varying identification of a current condition or of each of multiple applicable conditions and by updating time-varying versions of parameters at each time stamp. It will be appreciated that, during training, the value of changes of the learnable parameters can change at different steps of the algorithm (e.g., as determined using an update rule such as stochastic gradient descent).

At block 840, loss detector 735 calculates a loss function using the predicted results and known results. The independent variables of the loss function can be learnable parameters, and the dependent variable of the loss function can be a loss indicating a difference between predicted and actual results. Thus, the loss function can be used to estimate how well a model configured with current values for the learnable parameters is performing relative to a performance associated with different parameter values. In some instances, a predicted result includes multiple predicted results (corresponding to different outputs of the model), and the loss can be defined to be a weighted average based on the difference between the multiple predicted results and the corresponding actual results.

At block 845, a training controller determines whether training is complete. The determination may be based on whether a training-completion condition is satisfied. The training-completion condition may be configured to be satisfied when (for example) a predefined number of training iterations are completed, a predefined performance metric threshold (e.g., accuracy, precision, recall, etc.) is met, etc.

If training is not complete, process 800 proceeds to block 850, where value updator 725 updates parameter values and the parameter data structure. The update may be based on the predicted results, the actual results, and/or the calculated loss function. The update may be based on evaluating a gradient in a loss function, identifying a loss-reducing direction to “move” from a current parameter-value-associated position, and identifying new parameter values based on the loss-reducing direction.

Process 800 then returns to block 830 for a next training iteration.

If training was determined to be complete at block 845, process 800 proceeds to block 855, where the trained model is used to process non-training data. The non-training data may be live data, experimental data, etc.

A result of the model execution may be used to identify a real-world action that is to be taken (e.g., in a laboratory). For example, the model may be configured to predict whether or an extent to which a cell will grow or a likelihood that a cell will lyse given particular assumptions. As another example, the model may be configure to identify one or more particular parameter values that are predicted to result in a target result (e.g., one of two potential target results, a highest predicted target result, an above-threshold target result, etc.). In some instances, a method includes performing such an identified action. The real-world action may include (for example) performing a particular gene edit (e.g., so as to change, remove or replace a gene of a cell). The real-world action may include using a particular reagent in a reaction, adding a particular reaction to a solution, or using a particular buffer (e.g., for a cell culture).

### EXAMPLE

Millard’s model of central carbon metabolism (as described at Millard P, Smallbone K, Mendes P (2017) Metabolic regulation is sufficient for global and robust coordination of glucose uptake, catabolism, energy production and growth in Escherichia coli. PLoS Comput Biol 13(2): e1005396. https://doi.org/10.1371/journal.pcbi.1005396, which is hereby incorporated by reference in its entirety for all purposes) describes E. coli growing at steady state in a chemostat. Ordinary differential equations are used to describe biochemical reaction rates as a function of substrate, product, and allosteric effector concentrations. The advantage of this approach over other methods (like flux-balance analysis) is that gene expression and metabolic regulation like allostery is directly modeled.model is a [describe advantages of model]. However, using this model to process specific types of data is challenging due to inconsistencies between the model’s structure and the types of data that may be available in various data sets (e.g., the model can’t be applied to E. coli growing in non-steady state conditions). Fitting this model to heterogeneous dataset is challenging: for example, ecoli growing at different growth rates or in different growth media express different quantities of enzymes, which affects their activity (in our model, parametrized by the various Vmax parameters).

One approach would be to fit the model independently to different datasets - for example, one version of the model could be fit to bacterias growing in exponential phase, one could be fit to bacterias growing in a glucose rich media, and so on. This approach would be simple - but - greatly reduce the data used to train the model.

In a training data set, each set of three consecutive rows corresponds to three replicate measurements of a single condition. A condition is characterized by the following factors:
A base strain, which has some deletions (e.g., ackA) with respect to the “wild-type” Millard model.
For each product of interest, a plasmid expressing an enzyme needed to produce that product.
A set of “valves” that have been turned off in the condition (i.e., eliminating the presence of an enzyme by degrading its mRNA and/or protein). These are chromosomal changes with respect to the background strain for each product.
For some conditions, a factor identifies an additional knockout or additional plasmid that is present.
The growth medium. For most measurements this is the same, with glucose as the carbon source. For the measurements of xylitol production, the medium also contains xylose.

The measurements we will fit are product fluxes (several different products are captured in the dataset).

Model Setup. An augmented version of the Millard model is fit to the training data set. The augmented version includes a single model representing all of the enzymes needed for all of the products. The default model configuration corresponds to the base strain, with any deletions relative to Millard specified by setting their Vₘₐₓ value to zero. All of the production enzymes are turned off (via setting corresponding parameter values to 0),, and parameter values and initial state values are set to represent the glucose-based medium with no additional components.

Parameter configuration. Some of the parameters in the Millard model is Vₘₐₓ. A different Vmax parameter is defined for each enzyme in the model. A modeling assumption is imposed that Vₘₐₓ values do not differ between the glucose and glucose+xylose media. Thus, each of the Millard Vₘₐₓ parameters is represented as a global parameter:
param_e1_vmax = LearnableParameter(
name=‘E1_Vmax_global’,
variable=TargetVariable(model_parameter=‘E1_Vmax’),
global_param=GlobalParameter())

The Vₘₐₓ parameters of the plasmid-expressed “production” enzymes are represented as shared parameters, e.g. (for citramalate production):
param_cimA_vmax = LearnableParameter(
name=‘cimA_Vmax_shared’,
variable=TargetVariable(model_parameter=‘cimA_Vmax’),
shared _param=SharedParameter(conditions=[...]))

The condition list is populated from the data spreadsheet by extracting the conditions where the pSMART-cimA plasmid is included in the background strain.

Conditional assignments. To generate the description of parameter assignments specific to each condition, the appropriate valves by setting the Vₘₐₓ parameters of the corresponding enzymes were set to zero. E.g., to turn off the G and Z valves:
cond_gz_off = [VariableAssignment(variable=TargetVariable(model_parameter=‘G_Vmax’), value=0.0),
VariableAssignment(variable=TargetVariable(model_parameter=‘Z_Vmax’), value=0.0)]

These valve settings are populated from the valve_p and valve_g columns of the sheet by mapping the valve letters to VariableAssignments for the corresponding enzymes in the Millard model.

Use Case. Initial Chemostat Runs (wildtype, glucose, 5 growth rates). A model is configured o simulate wild-type E. coli in glucose medium at five different growth rates. The Millard model parameters will fall into three buckets:
Default parameters that will not be learned: parameters from the literature;
Global parameters: parameters such as kinetic turnover rates (Kcats) and Michaelis constants (Kms) that are not expected to be condition-specific
Local parameters: condition-specific parameters such as Vₘₐₓ parameters, for which a distinct value will be learned for each condition.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be rearranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
defining a set of conditions to be simulated via execution of a machine-learning model;
identifying, for each condition of the set of conditions, a set of learnable condition-specific parameters to configure a model architecture used for the condition;
stipulating that a first learnable condition-specific parameter associated with a first condition of the set of conditions is a shared or global parameter that is to have a same value as at least one other learnable condition-specific parameter, wherein each of the at least one other learnable condition-specific parameter is associated with a corresponding other condition of the set of conditions;
configuring one or more parameter data structures with parameter values for the sets of condition-specific parameters for the sets of conditions, wherein the configuration imposes a constraint that a value for the first condition-specific parameter and the at least one value for the at least one other condition-specific parameter are the same as each other;
training the machine-learning model using the configured one or more parameter data structures; and
executing the trained machine-learning model by processing another data set.

2. The method of claim 1, wherein configuring the one or more parameter data structures comprises:
generating an initial version of a parameter data structure of the one or more parameter data structures to include a value for each of the sets of learnable condition-specific parameters of the set of conditions;
identifying an initial value to initially define the shared or global parameter; and
generating a modified version of the parameter data structure to replace an initial version of the at least one other learnable condition-specific parameter with the initial value.

3. The method of claim 1, wherein training the machine-learning model is performed using a loss function that relates loss to values of a set of unique learnable parameters, and wherein the quantity of unique learnable parameters in the set of unique learnable parameters is less than a quantity of parameters represented in the one or more parameter data structures.

4. The method of claim 1, wherein training the machine-learning model includes:
calculating a loss function, wherein the loss function associates a particular loss with values of a set of learnable parameters, wherein the set of learnable parameters includes a particular learnable parameter corresponding to the first learnable condition-specific parameter and the at least one other learnable condition-specific parameter;
identifying a new set of values for the set of learnable parameters using the loss function, wherein the new set of values includes a new value for the particular learnable parameter; and
updating the one or more parameter data structures using the new set of values for the set of learnable parameters, wherein the updating includes setting each of the at least one value for the at least one other condition-specific parameter and the value for the first condition-specific parameter to the new value.

5. The method of claim 1, wherein the first learnable condition-specific parameter is a shared parameter, and wherein the combination of the first condition and each corresponding other condition associated with the at least one other learnable condition-specific parameter are an incomplete subset of the set of conditions, and wherein the method further comprises:
stipulating that a different learnable condition-specific parameter is a global parameter that is to have a same value across all conditions in the set of conditions;
wherein configuring the one or more parameter data structures imposes a constraint that values for parameters corresponding to the global parameter are to be the same across conditions.

6. The method of claim 1, wherein the machine-learning model is a model to simulate a biological cell, and wherein at least one of the set of conditions corresponds to a simulation where a particular gene is missing or inactive.

7. The method of claim 1, wherein the machine-learning model is a model to simulate a biological cell, and wherein at least one of the set of conditions corresponds to a simulation where a particular reagent is added to a medium external to the biological cell.

8. The method of claim 1, further comprising:
determining, based on a result of the execution of the trained machine-learning model, a gene edit to make or a reagent to use; and
implementing a real-world action in a laboratory environment that includes making the gene edit or using the reagent.

9. A system comprising:
one or more data processors; and
a non-transitory computer readable storage medium containing instructions which, when executed on the one or more data processors, cause the one or more data processors to perform a set of actions including:
defining a set of conditions to be simulated via execution of a machine-learning model;
identifying, for each condition of the set of conditions, a set of learnable condition-specific parameters to configure a model architecture used for the condition;
stipulating that a first learnable condition-specific parameter associated with a first condition of the set of conditions is a shared or global parameter that is to have a same value as at least one other learnable condition-specific parameter, wherein each of the at least one other learnable condition-specific parameter is associated with a corresponding other condition of the set of conditions;
configuring one or more parameter data structures with parameter values for the sets of condition-specific parameters for the sets of conditions, wherein the configuration imposes a constraint that a value for the first condition-specific parameter and the at least one value for the at least one other condition-specific parameter are the same as each other;
training the machine-learning model using the configured one or more parameter data structures; and
executing the trained machine-learning model by processing another data set.

10. The system of claim 9, wherein configuring the one or more parameter data structures comprises:
generating an initial version of a parameter data structure of the one or more parameter data structures to include a value for each of the sets of learnable condition-specific parameters of the set of conditions;
identifying an initial value to initially define the shared or global parameter; and
generating a modified version of the parameter data structure to replace an initial version of the at least one other learnable condition-specific parameter with the initial value.

11. The system of claim 9, wherein training the machine-learning model is performed using a loss function that relates loss to values of a set of unique learnable parameters, and wherein the quantity of unique learnable parameters in the set of unique learnable parameters is less than a quantity of parameters represented in the one or more parameter data structures.

12. The system of claim 9, wherein training the machine-learning model includes:
calculating a loss function, wherein the loss function associates a particular loss with values of a set of learnable parameters, wherein the set of learnable parameters includes a particular learnable parameter corresponding to the first learnable condition-specific parameter and the at least one other learnable condition-specific parameter;
identifying a new set of values for the set of learnable parameters using the loss function, wherein the new set of values includes a new value for the particular learnable parameter; and
updating the one or more parameter data structures using the new set of values for the set of learnable parameters, wherein the updating includes setting each of the at least one value for the at least one other condition-specific parameter and the value for the first condition-specific parameter to the new value.

13. The system of claim 9, wherein the first learnable condition-specific parameter is a shared parameter, and wherein the combination of the first condition and each corresponding other condition associated with the at least one other learnable condition-specific parameter are an incomplete subset of the set of conditions, and wherein the set of actions further comprises:
stipulating that a different learnable condition-specific parameter is a global parameter that is to have a same value across all conditions in the set of conditions;
wherein configuring the one or more parameter data structures imposes a constraint that values for parameters corresponding to the global parameter are to be the same across conditions.

14. The system of claim 9, wherein the machine-learning model is a model to simulate a biological cell, and wherein at least one of the set of conditions corresponds to a simulation where a particular gene is missing or inactive.

15. The system of claim 9, wherein the machine-learning model is a model to simulate a biological cell, and wherein at least one of the set of conditions corresponds to a simulation where a particular reagent is added to a medium external to the biological cell.

16. A computer-program product tangibly embodied in a non-transitory machine-readable storage medium, including instructions configured to cause one or more data processors to perform a set of actions including:
defining a set of conditions to be simulated via execution of a machine-learning model;
identifying, for each condition of the set of conditions, a set of learnable condition-specific parameters to configure a model architecture used for the condition;
stipulating that a first learnable condition-specific parameter associated with a first condition of the set of conditions is a shared or global parameter that is to have a same value as at least one other learnable condition-specific parameter, wherein each of the at least one other learnable condition-specific parameter is associated with a corresponding other condition of the set of conditions;
configuring one or more parameter data structures with parameter values for the sets of condition-specific parameters for the sets of conditions, wherein the configuration imposes a constraint that a value for the first condition-specific parameter and the at least one value for the at least one other condition-specific parameter are the same as each other;
training the machine-learning model using the configured one or more parameter data structures; and
executing the trained machine-learning model by processing another data set.

17. The computer-program product of claim 16, wherein configuring the one or more parameter data structures comprises:
generating an initial version of a parameter data structure of the one or more parameter data structures to include a value for each of the sets of learnable condition-specific parameters of the set of conditions;
identifying an initial value to initially define the shared or global parameter; and
generating a modified version of the parameter data structure to replace an initial version of the at least one other learnable condition-specific parameter with the initial value.

18. The computer-program product of claim 16, wherein training the machine-learning model is performed using a loss function that relates loss to values of a set of unique learnable parameters, and wherein the quantity of unique learnable parameters in the set of unique learnable parameters is less than a quantity of parameters represented in the one or more parameter data structures.

19. The computer-program product of claim 16, wherein training the machine-learning model includes:
calculating a loss function, wherein the loss function associates a particular loss with values of a set of learnable parameters, wherein the set of learnable parameters includes a particular learnable parameter corresponding to the first learnable condition-specific parameter and the at least one other learnable condition-specific parameter;
identifying a new set of values for the set of learnable parameters using the loss function, wherein the new set of values includes a new value for the particular learnable parameter; and
updating the one or more parameter data structures using the new set of values for the set of learnable parameters, wherein the updating includes setting each of the at least one value for the at least one other condition-specific parameter and the value for the first condition-specific parameter to the new value.

20. The computer-program product of claim 16, wherein the first learnable condition-specific parameter is a shared parameter, and wherein the combination of the first condition and each corresponding other condition associated with the at least one other learnable condition-specific parameter are an incomplete subset of the set of conditions, and wherein the set of actions further comprises:
stipulating that a different learnable condition-specific parameter is a global parameter that is to have a same value across all conditions in the set of conditions;
wherein configuring the one or more parameter data structures imposes a constraint that values for parameters corresponding to the global parameter are to be the same across conditions.

21. The computer-program product of claim 16, wherein the machine-learning model is a model to simulate a biological cell, and wherein at least one of the set of conditions corresponds to a simulation where a particular gene is missing or inactive..
